(19) United States
(12) Patent Application Publication
(10) Pub. No.: US 2013/0317318 A1
(43) Pub. Date: Nov. 28, 2013

(54) METHODS AND DEVICES FOR ACQUIRING ELECTRODERMAL ACTIVITY

(71) Applicant: QUALCOMM INCORPORATED, San Diego, CA (US)

(72) Inventors: Robert S. TARTZ, San Diego, CA (US); Jay S. KING, San Diego, CA (US); Aniket A. VARTAK, San Diego, CA (US)

(73) Assignee: QUALCOMM Incorporated, San Diego, CA (US)

(21) Appl. No.: 13/692,363

(22) Filed: Dec. 3, 2012

Related U.S. Application Data
(60) Provisional application No. 61/651,955, filed on May 25, 2012.

(51) Int. Cl. A61B 5/053; A61B 5/11; A61B 5/00

(52) U.S. Cl. CPC A61B 5/0531; A61B 5/7225; A61B 5/1125; A61B 5/7221; A61B 5/6898
USPC 600/301; 600/393

(57) ABSTRACT

Handheld devices using an array of stainless steel electrodes located on an edge and/or back of the handheld devices for acquiring electrodermal activity are provided. The stainless steel electrode array may allow for the skin conductance level (SCL) or skin conductance response (SCR) on an individual to be measured and collected. The skin conductance signal may be related to sympathetic nervous system activity which is a major component of human emotion, known as arousal, or emotional intensity such as anxiety, stress, fear, or excited, etc.

### CROSS-REFERENCE TO RELATED APPLICATIONS

The present application for patent claims priority to Provisional Application No. 61/651,955 entitled “METHODS AND DEVICES FOR ACQUIRING ELECTRODERMAL ACTIVITY ON A HANDHELD DEVICE USING STAINLESS STEEL ELECTRODES” filed May 25, 2012, and assigned to the assignee hereof and hereby expressly incorporated by reference herein.

### BACKGROUND

1. Technical Field

Aspects of the present disclosure relate generally to methods and devices for acquiring electrodermal activity.

2. Background

Electrodermal activity (EDA) is measured in microsiemens (μS) and is a term that refers to how well the skin conducts electricity when an external direct current (DC) or constant voltage is applied. That is, the EDA measures the electrical conductance of the skin of an individual, which varies with its moisture level from sweat emanating from the eccrine sweat glands that are found all over the body but most dense on the palms of the hands and soles of the feet. Electrodermal activity (EDA) is also known as skin conductance, galvanic skin response (GSR), electrodermal response (EDR), psychogalvanic reflex (PGR) and skin conductance response (SCR).

Standard silver-silver chloride (Ag/AgCl) electrodes are typically used for measurement of electrodermal activity and other biopotential signals since they are practically non-polarizable.

Currently electrodermal recording devices exist that are used in laboratory settings for measuring electrodermal activity. All devices currently on the market consist of some type of wearable electrodes, typically fixed to the distal or medial phalanges of the first two fingers (e.g., Thought Technology™) of the individual, for measuring the electrodermal activity. Another wearable device currently on the market is the Affectiva Q Sensor™ which attaches to the wrist of the individual.

However, no device exists that can be gripped by an individual to accurately measure electrodermal activity which could be deployed in a handheld form factor. Applications requiring reliable measurement of electrodermal activity (EDA) from the surface of a handheld device would require dry, reusable electrodes that are durable and malleable around curved surfaces. While it may be possible to use sintered Ag/AgCl electrodes in such a device, they are somewhat expensive and their durability and malleability are questionable. It may also be possible to use common stainless steel electrodes as they are very cost effective, however, stainless steel electrodes perform poorly when passing DC currents as they easily polarize.

There are three (3) major obstacles to designing a handheld device for measuring electrodermal activity. These obstacles include the material of the electrode, the configuration of the electrode and the grip force, as changing the grip force and grip force that is too firm can result in distortion of the electrodermal signal.

### SUMMARY

The following presents a simplified summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In one aspect, the disclosure provides a device, such as a mobile phone, for acquiring electrodermal activity. The device may comprise an array of stainless steel electrodes located on the edges and/or back of the device for the acquiring electrodermal activity of an individual holding the device. A polarity switching module may be coupled to the array of stainless steel electrodes for switching polarity of electrodes in the array of stainless steel electrodes to prevent polarization of the stainless steel electrodes for skin conductance measurements. The device may also include a memory device that may include operations (instructions) for storing received input (or incoming) signals and/or feedback signals from the array of stainless steel electrodes (i.e. electrodermal activity data).

At least one processor may be coupled to the array of stainless steel electrodes and the memory device and configured to determine a number of adjacent electrode pairs in the array of stainless steel electrodes that have come into contact with the skin, such as the hands, of an individual to scale a skin conductance response threshold. Next, the processor may be configured to fuse all the negative electrodes together and all positive electrodes together in the array of stainless steel electrodes upon activation of the electrodes in the array of stainless steel electrodes. The electrodes in the array of electrodes are activated upon the electrodes on the device becoming active and alternating in polarity, e.g. + − + − + − + − . To alternate polarity, the current flow direction through the one or more electrode pairs in the array of stainless steel electrodes may be reversed as each electrode pair becomes active. The processor may then be configured to measure a single overall skin conductance response to capture a total electrode activity measurement and automatically adjust the skin conductance response threshold to count legitimate skin conductive responses using the number of contacted electrode pairs, wherein the counted legitimate skin conductive responses is a determination of arousal.

In one example, the total electrodermal activity measurement measures a reaction of an individual to an advertisement that has appeared on the device. In another example, the total electrodermal activity measurement may be used to track stress levels of an individual. A graph of the total electrodermal activity measurement captured over time may be generated and an index of emotional arousal based on historical data may be computed.

In another aspect, the device may also include a force sensor array coupled to each electrode pair in the array of stainless steel electrodes for detecting grip force. Grip force is the force that may temporarily be applied by an individual to the stainless steel electrodes on the device. Changing grip force or applying too much grip force can result in distortion of the electrodermal signal on the device which in turn may create false-positive and false-negative artifacts in the data. Using data obtained from the force sensor array, the at least one processor may be further configured to invalidate captured electrodermal activity data if the grip force changes or if the grip force exceeds a grip force threshold.

The array of stainless steel electrodes may be embedded on a right side and a left side of the device where the array of stainless steel electrodes is interleaved down the sides and back of the device. The array of stainless steel electrodes may also be embedded on an upper edge portion and a lower edge portion wrapping around to a backside of the device.

In yet another aspect, the disclosure provides a method for acquiring electrodermal activity on a device using an array of stainless steel electrodes embedded on the device. The method may include determining a number of adjacent electrode pairs in the array of stainless steel electrodes contacted to scale a skin conductance response threshold; fusing all negative electrodes together and all positive electrodes together in the array of stainless steel electrodes upon activation of electrodes in the array of stainless steel electrodes; measuring a single overall skin conductance response to capture a total electrode activity measurement; and automatically adjusting the skin conductance response threshold to count legitimate skin conductance responses using the number of contacted electrode pairs, where the counted legitimate skin conductive responses is a determination of arousal.

In one example, the method may further comprise detecting grip force from the temporary gripping of the one or more electrode pairs in the array of stainless steel electrodes and invalidating captured electrodermal activity data if the grip force changes or if the grip force exceeds a grip force threshold. Additionally, the method may comprise reversing the current flow direction through the one or more electrode pairs in the array of stainless steel electrodes as each electrode pair is activated; generating a graph of the total electrodermal activity measurement captured over a period of time; and computing an index of emotional arousal based on historical data.

In yet another aspect, the disclosure provides a device, such as a mobile phone, for acquiring electrodermal activity where the device comprises means for determining a number of adjacent electrode pairs in an array of stainless steel electrodes contacted to scale a skin conductance response threshold; means for fusing all negative electrodes together and all positive electrodes together in the array of stainless steel electrodes upon activation of electrodes in the array of stainless steel electrodes; and means for measuring a single overall skin conductance response to capture a total electrode activity measurement. The device may further comprise means for detecting a grip force change from the temporary gripping of the one or more electrode pairs in the array of stainless steel electrodes and means for invalidating captured electrodermal activity data if the grip force changes or if grip force exceeds a grip force threshold.

The device may further comprise means for reversing the current flow direction through the one or more electrode pairs in the array of stainless steel electrodes as each electrode pair is activated; means for generating a graph of the total electrodermal activity measurement captured over a period of time; and means for computing an index of emotional arousal based on historical data. Additionally, the device may comprise means for automatically adjusting the skin conductance response threshold to count legitimate skin conductance responses using the number of contacted electrode pairs, where the counted legitimate skin conductive responses is a determination of arousal.

These and other aspects of the disclosure will become more fully understood upon a review of the detailed description, which follows.

### BRIEF DESCRIPTION OF THE DRAWINGS

The accompanying drawings, together with the specification, illustrate exemplary embodiments of the present invention, and, together with the description, serve to explain the principles of the present disclosure.

FIG. 1 illustrates a model of ion/electron exchange that occurs in a pair of silver-silver chloride (Ag/AgCl) electrodes.

FIG. 2 illustrates an electrical model of a pair of stainless steel electrodes.

FIG. 3A illustrates the polarization effect during simultaneous skin conductance measurement from a pair of stainless steel electrodes gripped by an individual as compared to a wearable pair of standard Ag/AgCl electrodes, according to a first example.

FIG. 3B illustrates the polarization effect during simultaneous skin conductance measurement from a pair stainless steel electrodes gripped by an individual as compared to a wearable pair of standard Ag/AgCl electrodes, according to a second example.

FIG. 4 is a high level block diagram illustrating two (2) Ag/AgCl electrodes with no polarity switching.

FIG. 5 is a high level block diagram illustrating two (2) stainless steel electrodes with polarity switching.

FIG. 6 illustrates an example of the internal structure of the electrode switch network of FIG. 5 for switching the polarity of electrodes over time at regular intervals.

FIG. 7 is a high level block diagram illustrating a stainless steel electrode array with polarity switching.

FIG. 8 is a low level block diagram of the stainless steel electrode array of FIG. 7 with polarity switching.

FIG. 9A is a graph illustrating the measurement of skin conductance using a pair of standard Ag/AgCl electrodes secured to fingers of an individual, according to one example.

FIG. 9B is a graph illustrating skin conductance data collected concurrently with reference electrodes by gripping a pair of stainless steel electrodes located on a handheld device, with switching the polarity of the electrodes over time, according to one example.

FIG. 10A illustrates the effects of electrode polarization of stainless steel electrodes gripped on a handheld device when no polarity switching is implemented, as shown by the sharp drop in skin conductance level.

FIG. 10B illustrates the simultaneous data collected gripped on a handheld device with the polarity switching circuit, showing a clear skin conductance signal with no drop in skin conductance level.

FIG. 11A illustrates a front view of a partial handheld device having an interleaved electrode array layout according to one example.

FIG. 11B illustrates the back view of the handheld device of FIG. 11A.

FIG. 11C illustrates a side view of the handheld device for FIG. 11A with a first electrode pair activated.

FIG. 11D illustrates a side view of the handheld device for FIG. 11A with a second electrode pair activated.

FIG. 12 is a low level block diagram of the stainless steel electrode array with yjr polarity switching of FIG. 7 showing the fusing of the electrode pairs.

FIG. 13A illustrates a back view of a partial handheld device having an interleaved electrode array layout on the back of the device sampling a first set of electrodes according to one example.

FIG. 13B illustrates the back view of the handheld device of FIG. 13A sampling a second set of electrodes.

FIG. 14A illustrates a back view of a partial handheld device having an interleaved electrode array layout on a bottom edge portion of the device sampling a first set of electrodes according to one example.

FIG. 14B illustrates the back view of the partial handheld device of FIG. 14A, rotated 180 degrees, having an interleaved electrode array layout on a top edge portion of the device sampling a first set of electrodes according to one example.

FIG. 14C illustrates the back view of the partial handheld device of FIG. 14A sampling a second set of electrodes according to one example.

FIG. 14D illustrates the back view of the partial handheld device of FIG. 14B sampling a second set of electrodes according to one example.

FIG. 15 is a graph illustrating the effects of various static grip forces applied to Ag/AgCl electrodes on the skin conductance signal.

FIG. 16 is a graph illustrating the effects of dynamic grip force applied to Ag/AgCl electrodes on the skin conductance signal.

FIG. 17 illustrates a side view of a handheld device showing force sensors placed directly under each of the electrode pairs.

FIG. 18 illustrates a block diagram of an internal structure of an interactive handheld device, according to one example.

FIG. 19 illustrates a flow diagram of a method, which may be operational on an interactive handheld device, for acquiring electrodermal activity according to one example.

FIG. 20 illustrates a flow diagram of a method, which may be operational on an interactive handheld device, for acquiring electrodermal activity according to one example.

Elements and steps in the figures are illustrated for simplicity and clarity and have not necessarily been rendered according to any particular sequence. For example, steps that may be performed concurrently or in different order are illustrated in the figures to help to improve the understanding of various aspects of the disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

The word “exemplary” is used herein to mean “serving as an example, instance, or illustration.” Any implementation or embodiment described herein as “exemplary” is not necessarily to be construed as preferred or advantageous over other embodiments. Likewise, the term “embodiments” does not require that all embodiments include the discussed feature, advantage or mode of operation.

The term “handheld device” may refer to a mobile device, a wireless device, a mobile phone, a mobile communication device, a user communication device, personal digital assistant, mobile palm-held computer, a laptop computer, remote control and/or other types of mobile devices typically carried by individuals and/or having some form of communication capabilities (e.g., wireless, infrared, short-range radio, etc.).

While the present disclosure is described primarily with respect to handheld devices, the present disclosure may be applied and adapted to various devices. The present disclosure may be applied to any type of device that can be gripped, held or come into contact with skin of an individual, including but not limited to, handle bars on exercise equipment, such as a treadmill, biofeedback therapy devices and user interfaces, such as a mouse for a computer, where there is a desire for measuring electrodermal activity. Also, a variety of other embodiments are contemplated having different combinations of the below described features of the present disclosure, having features other than those described herein, or even lacking one or more of those features. As such, it is understood that the disclosure can be carried out in various other suitable modes.

### Overview

Devices using stainless steel electrodes pairs located on the edges and/or back of the devices for acquiring electrodermal activity are provided. The stainless steel electrodes may allow for skin conductance or electrodermal activity (EDA) of an individual to be measured and collected. The polarity of the stainless steel electrodes pairs may change to prevent polarization of the stainless steel electrodes for skin conductance measurements. Electrodermal activity reflects sympathetic nervous system activation and is related to a major component of human emotion known as arousal (Boucsein, 1992). Emotional arousal is similar to emotional intensity which is orthogonal to emotional valence, the other major component in human emotion which EDA may not measure well. Valence is an evaluative component (e.g., positive, negative) as proposed by the circumplex model of affect (Russell, 1980). For example, high emotional arousal may be experienced in various emotional states, such as anxiety, stress, fear, or anger (which are negative states) or more positive states such as excitement.

According to one feature, the skin conductance data collected may be used for marketing purposes. For example, a handheld device may be used to sense how an individual reacts to an advertisement that has appeared on the handheld device. The individual may be provided with a discount or other reward to opt-in or participate in this feature.

According to another feature, the skin conductance data collected may be used with wireless health applications. For example, the handheld device may be used to track stress levels of an individual. The health application on the handheld device may use the collected data to generate a graph of the individual's skin conductance level over a specific time period, such as on a daily basis. The individual may then use this information in a biofeedback application, for example, to adjust their EDA downwards to a more relaxing state. Additionally, the skin conductance data collected may be shared with medical professionals.

According to another feature, the collected skin conductance data may be used in a variety of other applications. For example, collected skin conductance data may be used in connection with gaming to determine the emotions, emotional state or emotional arousal of the individual or that of competing players. For example, the game may take input for one or more individuals' emotional state. The emotional state of the one or more individuals may make inferences about the individuals. If emotional arousal is increasing, it may be inferred that the individual is excited by the game or is getting so aroused that the individual is not doing well and the game may automatically become easier by shifting to a different, easier level. Conversely, if the data indicates the individual is bored, the game may automatically become more difficult. That is, the data may be used as a feedback loop that allows the difficulty of the game to be adjusted in real time.

The collected skin conductance data may also be used in connection with social networking. When an individual is logged onto his/her social network page, such as Facebook®, using a handheld device, the collected skin conductance data may be used to update the status of the individual on social network page (e.g., the individual is stressed out). In other words, the collected skin conductance data may be used as a user interface enhancement or for contextual awareness, similar to that of gaming as described above. Based on the data, the user interface may become more engaging or less engaging or stimulating.

### Electrode Material:

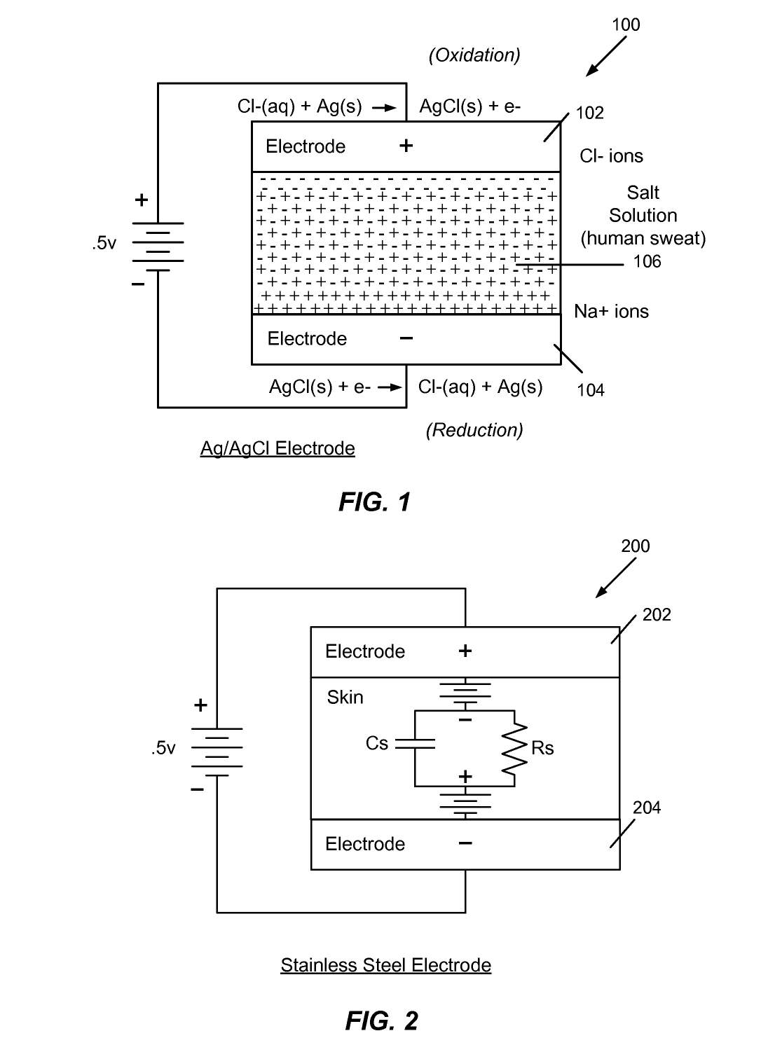

To collect skin conductance data, electrodes may be located on the edges and/or back of the handheld devices such that when an individual temporarily grips the device the skin conductance of the individual is easily measured. As discussed above, standard silver-silver chloride (Ag/AgCl) electrodes are typically used for acquiring electrodermal activity and other biopotential signals (e.g., electrocardiogram (ECG), electromyography (EMG)). FIG. 1 illustrates a model 100 of a pair of Ag/AgCl electrodes. The pair of Ag/AgCl electrodes may comprise a positive Ag/AgCl electrode 102 and a negative Ag/AgCl electrode 104.

As shown, a salt solution 106, such as a 1% salt solution found in human sweat, may be located between a positive (+) Ag/AgCl electrode and a negative (−) Ag/AgCl electrode. The salt solution 106 may be aqueous sodium chloride (NaCl), which contains both sodium ions (Na+) and chloride ions (Cl−) ions. When a small direct current (DC) voltage is applied to Ag/AgCl electrodes (e.g., +0.5 v DC typically to measure skin conductance), the Chloride on the negative electrode 104 dissolves and the negatively charged Cl-ion migrates to the positive electrode 102 where it combines with silver (Ag) to form AgCl plus a free electron. Thus, Ag/AgCl operates as a transducer between ion flow in human sweat (NaCl) and electron flow in the circuit which allows skin conductance to be accurately calculated. Although Ag/AgCl electrodes work well, the sintered Ag/AgCl electrodes are very expensive and although the sintered Ag/AgCl electrodes are durable, they do not conform well to curved surfaces. With regard to the printed type of Ag/AgCl electrodes, the Ag/AgCl electrodes have a thin Ag/AgCl layer that will wear away after many uses and will also oxidize over time. Thus, the printed type of Ag/AgCl electrodes cannot be used on the housing of a device that will be repeatedly used, perhaps over a period of years.

FIG. 2 illustrates an electrical model 200 of a pair of stainless steel electrodes. Stainless steel is a desirable material to measure skin conductance since it is durable, non-corrosive and can be easily formed to the housing of a device. Furthermore, stainless steel is also very cost effective compared to sintered Ag/AgCl electrodes. There are many variations of stainless steel but the most common type, 18/8 steel, is generally composed of 65-74% Iron, 18% Chromium, 8% Nickel, 2% Manganese, <0.08% Carbon and traces of other elements. Although highly conductive, stainless steel does not contain elements which react well with the ions found in human sweat, thus causing an electrical double layer to form over time as ions pile up near the electrode surface.

As shown in FIG. 2, the pair of stainless steel electrodes may comprise a positive stainless steel electrode 202 and a negative stainless steel electrode 204. When an individual places a hand and/or fingers on the pair of stainless steel electrodes, skin 206 from the hand and/or fingers may be in contact with and located between the positive stainless steel electrode 202 and the negative stainless steel electrode 204. As shown in FIG. 2, a simplified model of skin tissue and eccrine sweat glands may be modeled with an R-C circuit comprising a resistor (R_{S}) in parallel with a capacitor (C_{S}). A small direct current (DC) voltage may be applied to the pair of stainless steel electrodes (+0.5 v DC typically) and the skin conductance is measured.

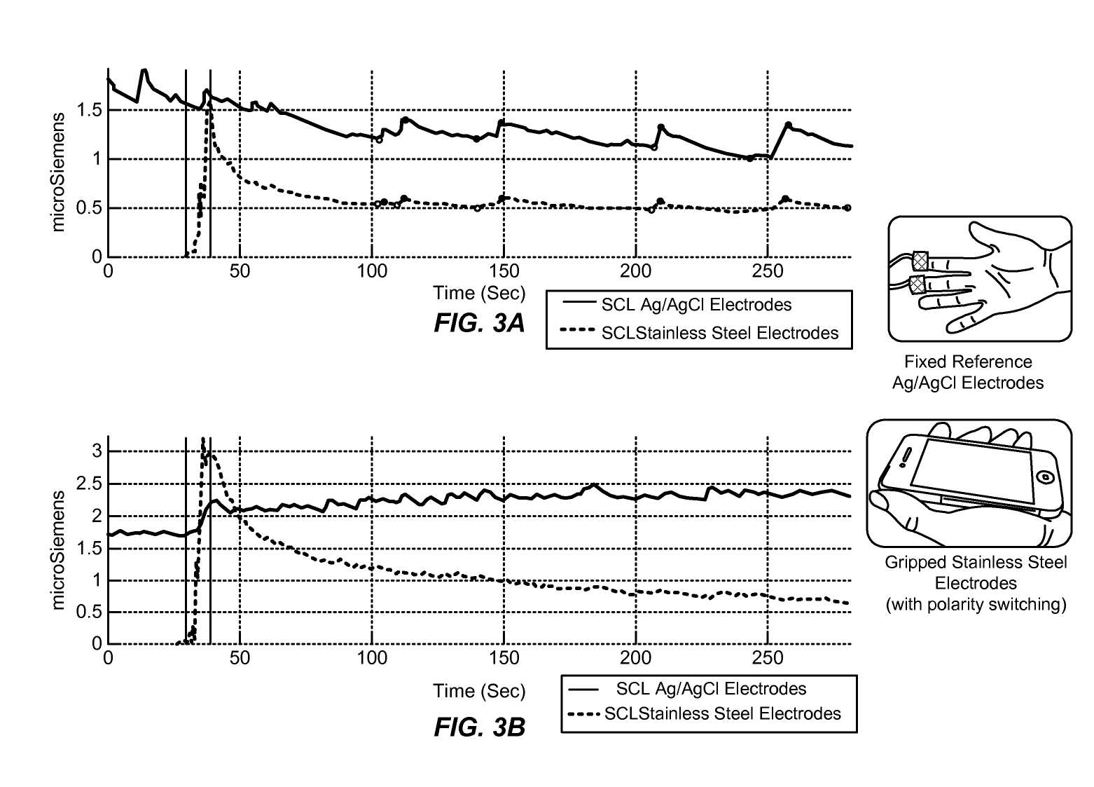

An electrical double layer may form after the electrodes are excited with a direct current and cause an error voltage to appear between the electrodes and skin that opposes the applied voltage. The net effect, known as “electrode polarization”, reduces current flow through the circuit and causes the calculated skin conductance to approach zero and be practically unusable. Electrode polarization on stainless steel begins very quickly and progressively increases. FIG. 3A illustrates the polarization effect during simultaneous skin conductance measurement from a pair of stainless steel electrodes gripped by an individual as compared to a wearable pair of standard Ag/AgCl electrodes, according to a first example. FIG. 3B illustrates the polarization effect during simultaneous skin conductance measurement from a pair of stainless steel electrodes gripped by an individual as compared to a wearable pair of standard Ag/AgCl electrodes, according to a second example. As shown in the figures, skin conductance level appears to dramatically fall shortly after gripping the stainless steel electrodes while the reference Ag/AgCl electrodes show the actual skin conductance level.

Moreover, the polarization effect may be dependent on the material of the electrodes. As shown in FIGS. 3A and 3B, the stainless steel electrodes may have a strong polarization effect. According to one embodiment, the fingers and/or palms of an individual's hand may be used to measure the skin conductance response as there is a high density of the eccrine sweat glands, which are known to be responsive to emotional and other psychological stimuli. As described in further detail below, the conductance may be measured by placing two electrodes next to the skin and passing a small electric current between the two points. When the individual experiences increased emotional arousal, his/her skin immediately becomes a slightly better conductor of electricity, due to hydration of skin with sweat and this response can then be measured and communicated.

According to one example, the polarity of the pair of stainless steel electrodes may be switched every 100 msec (10 Hz switching frequency) between +0.5 v and −0.5 v. Once the circuit and skin in contact with the pair of stainless steel electrodes has the opportunity to settle, the conductance measured during the +0.5 V state may be sampled, resulting in a final output sample rate of 5 samples per second.

As shown in both FIGS. 3A and 3B, the skin conductance signal from wearable Ag/AgCl reference electrodes illustrates the lack of the polarization effect while the skin conductance signal from gripped stainless steel electrodes on a mobile device illustrates the polarization effects when an individual is gripping a pair of stainless steel electrodes. One solution to the polarization problem that occurs with stainless steel electrodes may be to switch the polarity of the electrodes over time at regular intervals, as described below, thus keeping the electrical double layer from forming in the first place. Such a method may allow current to flow for a brief time while a sample is taken and then the polarity is reversed to allow current to flow in the opposite direction. Negatively charged Choloride ions, for example (Cl−), would not have enough time to pile up between the electrode and skin and cause an error voltage to form.

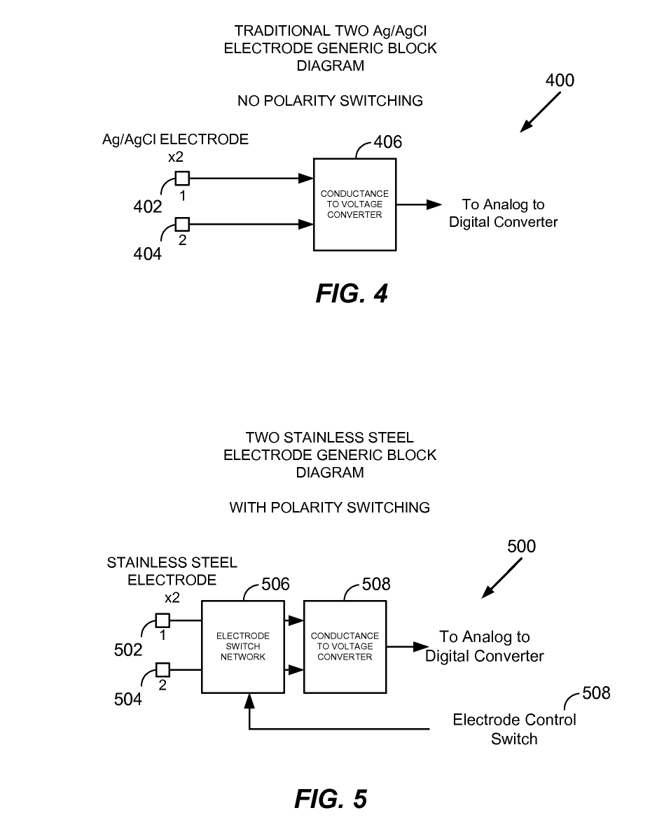

FIG. 4 is a high level block diagram 400 illustrating two (2) Ag/AgCl electrodes with no polarity switching. As shown, the two (2) Ag/AgCl electrodes 402, 404 may be connected to the input of a conductance to voltage converter 406 the output of which is sent to an analog to digital converter. The conductance from the electrodes is converted to a voltage which is then sent to an analog to digital converter. With no polarity switching, as described above, the stainless steel electrodes pairs may become polarized.

FIG. 5 is a high level block diagram 500 illustrating two (2) stainless steel electrodes with polarity switching. As shown, the two (2) stainless steel electrodes 502, 504 may be connected to the input of an electrode switch network 506 for switching polarity of the electrodes 502, 504. The electrode switch network 506 may be controlled by an electrode control switch 508. The output from the electrode switch network 506 may then be input into the conductance to voltage converter 508 which converts the conductance to a voltage which is then sent to an analog to digital converter

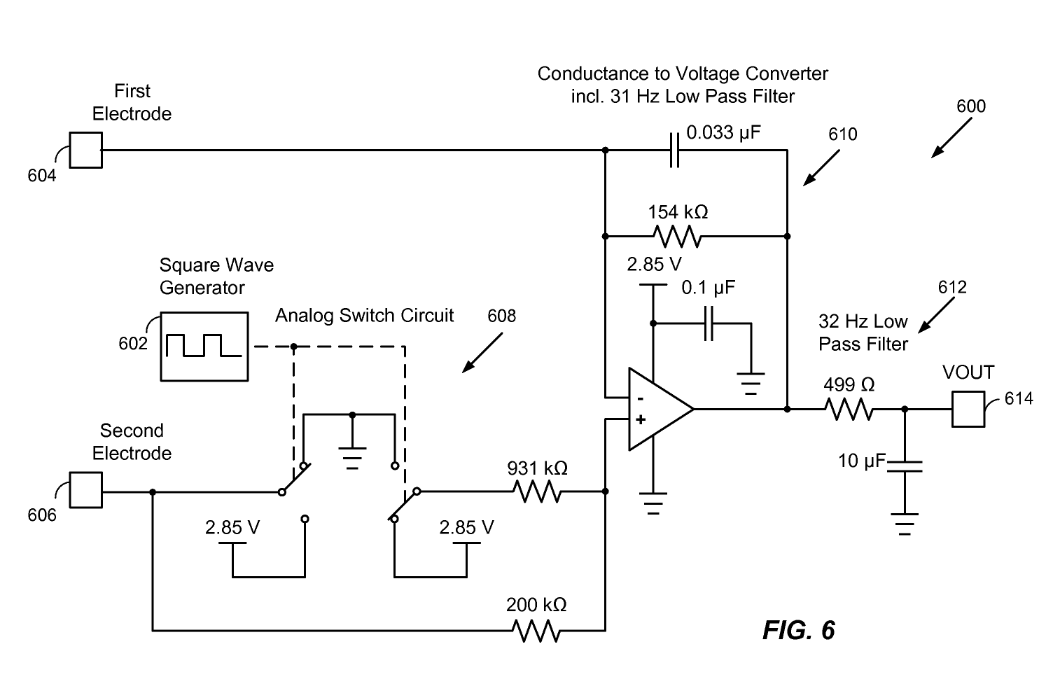

FIG. 6 illustrates an example of the internal structure of the electrode switch network of FIG. 5 for switching the polarity of electrodes over time at regular intervals. The electrode switch network 600 may provide a polarity switching system where the current flow direction through the skin is reversed in a periodic manner at regular intervals. A 50% nominal duty cycle square wave generator 602 may control the direction of current flow between a first electrode 604 and a second electrode 606 via an analog switch circuit 608. The voltage between the first electrode 604 and the second electrode 606 may be a nominal value, such as +0.5 v, depending on the polarity of the square wave. A switching frequency appropriate for a skin conductance signal, an inherently slow varying signal, may be selected. A conductance to voltage converter 610 (the op-amp circuit) may generate a voltage that is linearly proportional to the skin conductance presented between the first electrode 604 and the second electrode 606. The voltage may pass through a 32 Hz low pass filter 612 and then in a data extraction phase, a set of equations, as is known in the art, may be used to transform the VOUT voltage signal 614 to a skin conductance reading in microSiemens units.

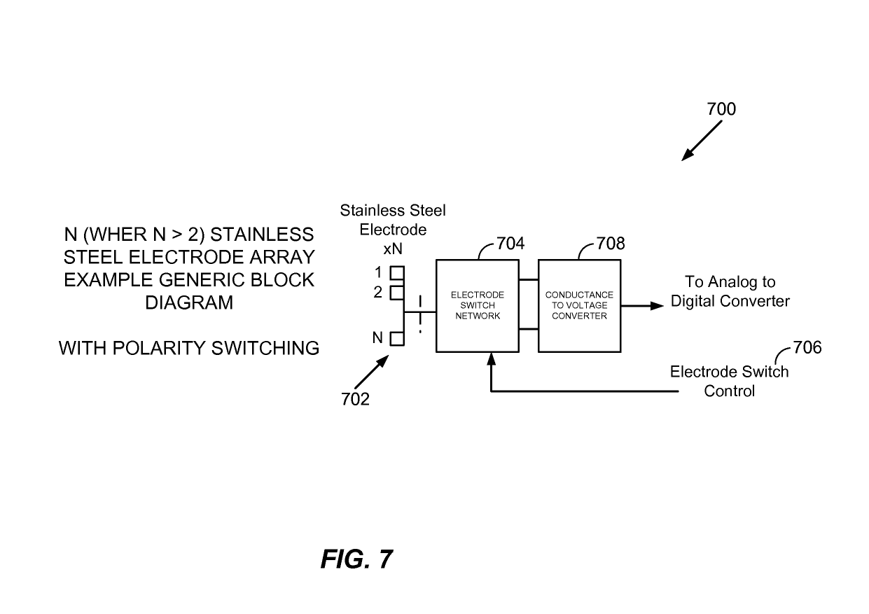

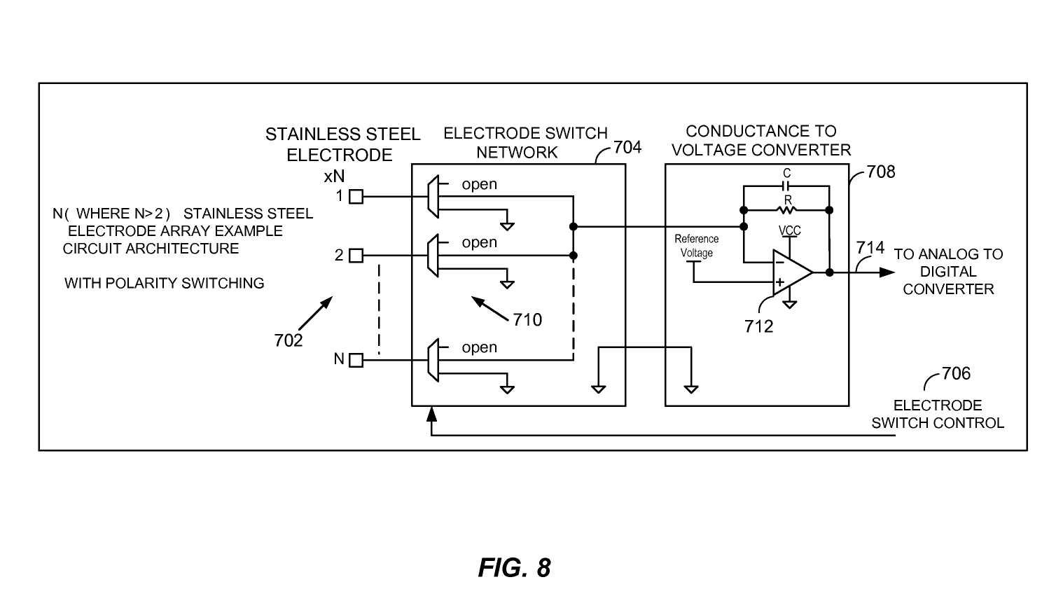

FIG. 7 is a high level block diagram 700 illustrating a stainless steel electrode array 702 with polarity switching. As shown, the stainless steel electrode array 702 may include N stainless steel electrodes where N>2. The stainless steel electrode array 702 may be connected to the input of an electrode switch network 704 for switching polarity of the electrodes. The electrode switch network 704 may be controlled by an electrode control switch 706. The output from the electrode switch network 704 may then be input into the conductance to voltage converter 708 which converts the conductance to a voltage which is then sent to an analog to digital converter.

FIG. 8 is a low level block diagram of the stainless steel electrode array of FIG. 7 with polarity switching. As shown and described above, the stainless steel electrode array 702 may include N stainless steel electrodes, where N>2, and may be connected to the input of the electrode switch network 700 to provide a polarity switching system where the current flow direction through the skin is reversed in a periodic manner at regular intervals. The electrode switch network 704, controlled by the electrode control switch 706, may include N switches 710 operable between open and closed positions, where N>2 is equal to the number of electrodes in the array 702. When in the open position, the output from the switches 710 may then be input into the conductance to voltage converter 708 to generate a voltage that is linearly proportional to the skin conductance. According to one embodiment, the conductance to voltage converter 708 may include an op amp 712 and the output from the switches 710 may be input into the inverting input of the op amp while the non-inverting input may be a reference voltage. An R-C circuit comprising a resistor (R) in parallel with a capacitor (C) may be in parallel with the inventing input of the op amp 712 and the output 714 of the op amp 712.

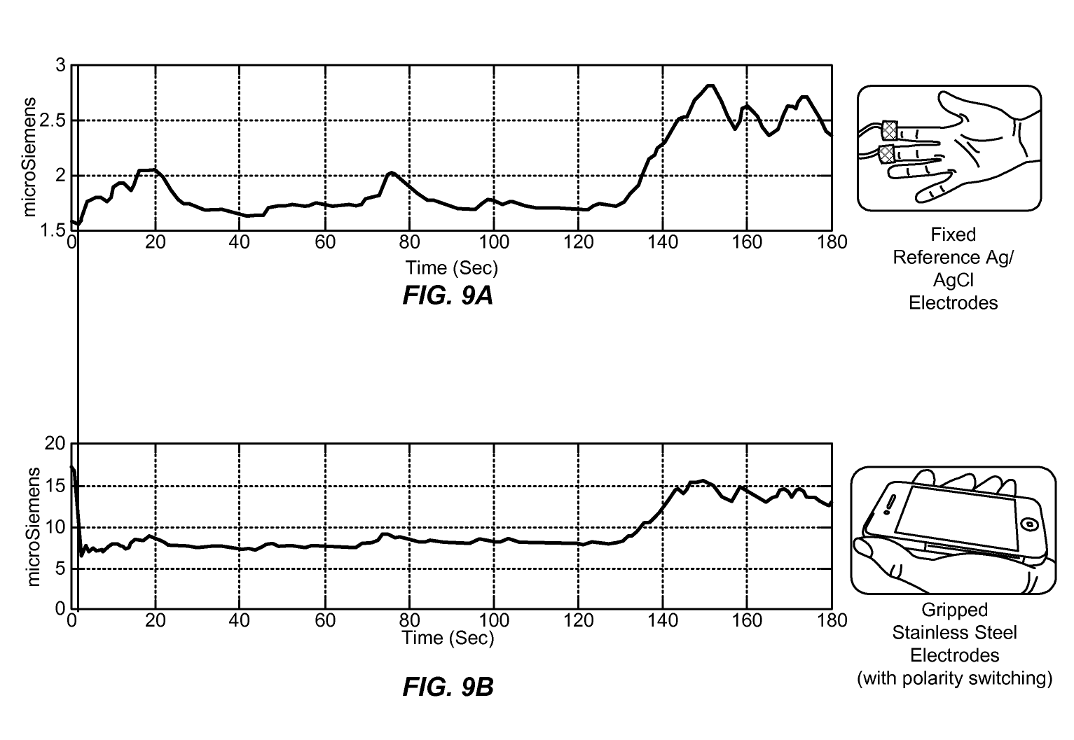

FIG. 9A is a graph illustrating the measurement of skin conductance using a pair of standard Ag/AgCl electrodes secured to fingers of an individual, according to one example. For example, the pair of Ag/AgCl electrodes may be attached to the index and middle fingers, respectively, of the individual and a small constant voltage applied. As shown, using the fixed pair of worn Ag/AgCl electrodes the skin conductance, measured in microSiemens units is constantly varying over time and a clean electrodermal signal can be measured/acquired.

FIG. 9B is a graph illustrating skin conductance data collected concurrently with reference electrodes by gripping a pair of stainless steel electrodes located on a handheld device, with switching the polarity of the electrodes over time, according to one example. As shown in FIG. 9B, the pair of gripped stainless steel electrodes, which switch polarity and sample electrodermal activity at pre-determined intervals when the polarity is positive, may allow for a clean electrodermal signal to be acquired without electrode polarization that may be highly correlated with another standard wearable reference sensor using a pair of Ag/AgCl electrodes (See FIG. 9A).

The data in the graphs of FIGS. 9A and 9B are correlated in that the data in each of the graphs was taken at a particular point in time with one particular individual. Data taken at other times with different individuals will be different.

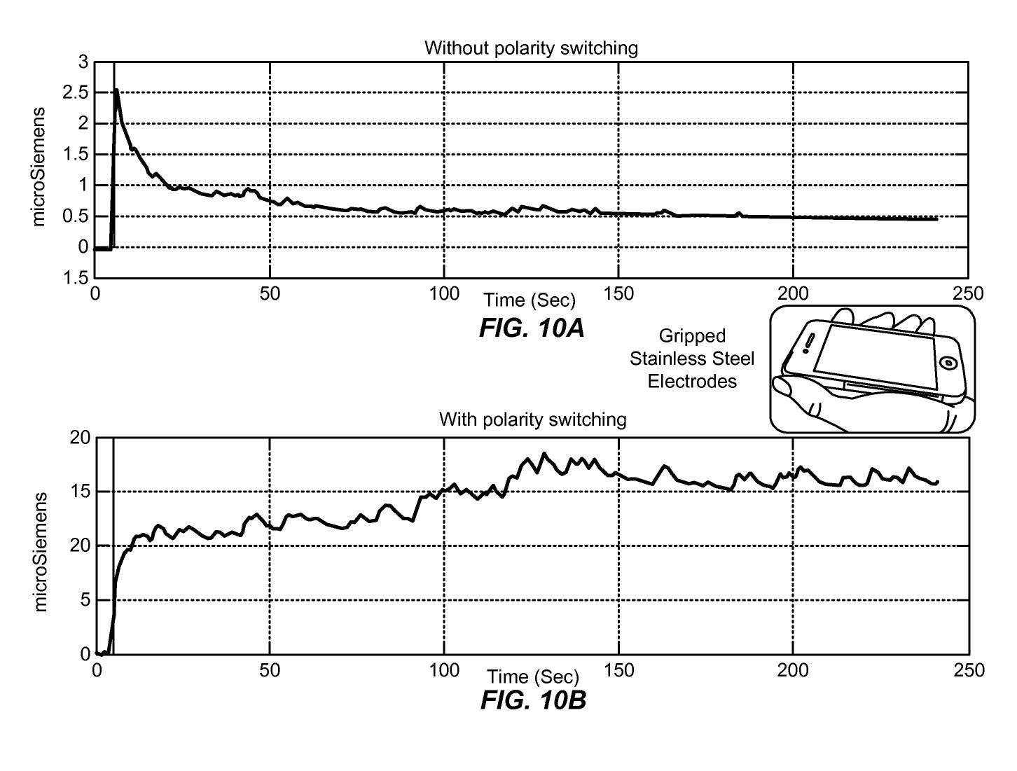

Using common stainless steel electrodes (without polarity switching) for skin conductance measurement has suggested that electrode polarization on stainless steel begins very quickly and progressively increases. By using the polarity switching circuit of FIG. 6 or 8 described above, charge accumulation and eventual polarization of stainless steel electrodes may be mitigated. FIG. 10A illustrates the effects of electrode polarization of stainless steel electrodes gripped on a handheld device when no polarity switching is implemented, as shown by the sharp drop in skin conductance level. Simultaneous data collected gripped on a handheld device with the polarity switching circuit is illustrated in FIG. 10B which shows a clear skin conductance signal with no drop in conductance level mitigating polarization from the stainless steel electrodes.

### Electrode Configuration:

Skin conductance may be dictated by the electrode (positive or negative) with the least amount of skin contact. As such, in one example, an electrode arrangement that allows for an even distribution of positive and negative electrode area contacted by the skin no matter how the device is gripped is provided. Furthermore, the arrangement of individual electrode segments and adjusting for the number of electrodes contacted may allow the sensor to be accurate regardless of how the device is being gripped. As such, the individual does not have to think where and how to grip the device.

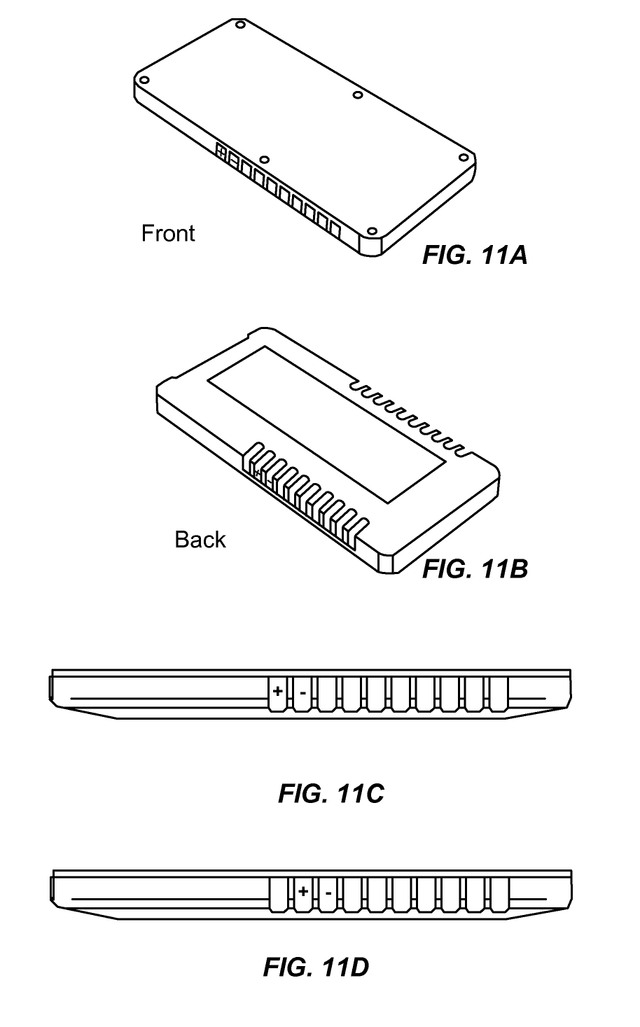

FIGS. 11A-11D illustrate a handheld device having an interleaved electrode array layout, according to one example. As shown, interleaving positive and negative electrode pairs down the sides of the device can maximize an even distribution of electrodes in contact with the skin. According to one example, making each electrode pair roughly the average size of a human fingertip may ensure an even contact area for positive and negative electrodes no matter how the device is contacted. In one embodiment each electrode pair may be approximately 1 cm across with at least 2 mm of space between electrodes for an accurate measurement of skin conductance, making each electrode about 4 mm across. As shown in FIGS. 11C and 11D, only a single electrode pair is activated at any one time. Sampling each electrode pair automatically reverses the polarity.

Additionally, since counting SCRs (skin conductance responses) is typically done by using an absolute threshold level for standard 1 cm diameter Ag/AgCl electrodes (typically 0.05 microsiemens), methods that can allow the threshold to adjust depending on how many positive/negative electrode pairs are contacted at any point in time as the device is gripped in different ways is provided.

Fusing Positive and Negative Electrodes

One method for allowing the skin conductance response threshold to adjust, depending on how many positive/negative electrode pairs are contacted at any point in time as the device is gripped in different ways, includes fusing positive electrodes in the array together and the negative electrodes in the array together. The method may briefly “scan” each adjacent electrode pair individually by sampling skin conductance for each electrode pair. If the skin conductance for an adjacent pair of electrodes exceeds a certain threshold value (e.g., 0.1 microsiemens), that pair of electrodes has been touched. The measured skin conductance is not being added together or totaled up; it is merely used to determine if an electrode pair has been touched.

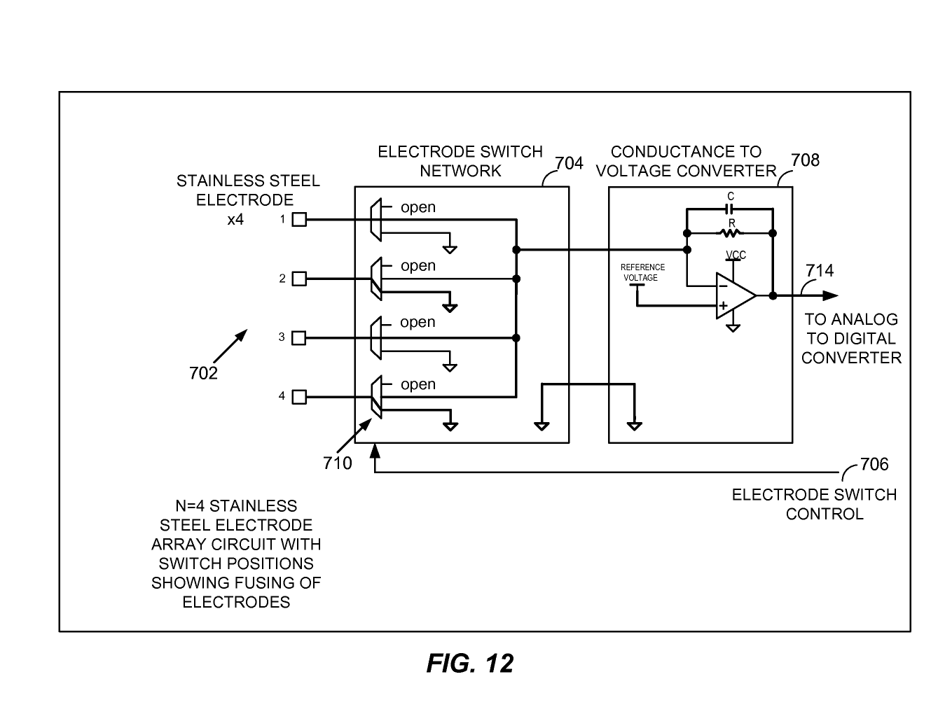

As each adjacent electrode pair is scanned, the electrode pair is activated. i.e., the electrodes on the device become active and alternate in polarity, e.g. + − + − + − + − . Next, all the positive electrodes are fused together (i.e. every other electrode in the array) and all the negative electrodes are fused together (i.e. every other electrode in the array). FIG. 12 is a low level block diagram of the stainless steel electrode array with polarity switching of FIG. 7 showing the fusing of the electrodes. Once all the positive electrodes are fused together and all the negative electrodes are fused together, a single overall skin conductance measurement may be taken to capture a total electrodermal activity measurement. The SCR threshold level may then be automatically adjusted based on number of electrodes contacted to determine if an SCR occurred. Such a strategy may also automatically reverse the polarity of each electrode as each immediately adjacent electrode pair is individually scanned.

### Combine Electrodermal Activity

One method for allowing the threshold to adjust, depending on how many positive/negative electrode pairs are contacted at any point in time as the device is gripped in different ways, includes combining the electrodermal activity data to determine a total electrodermal activity measurement. The method may briefly “measure” each adjacent electrode pair individually by sampling skin conductance for each pair. If a threshold is exceeded (e.g., 0.1 microsiemens), the electrode pair is determined as contacted and counted in a total of contacted electrode pairs and the skin conductance from each contacted pair is totaled for a total skin conductance level result. The SCR threshold level may then be adjusted based on the number of electrodes contacted to determine if an SCR occurred. Such a strategy may also automatically reverse the polarity of each electrode as each immediately adjacent electrode pair is individually scanned.

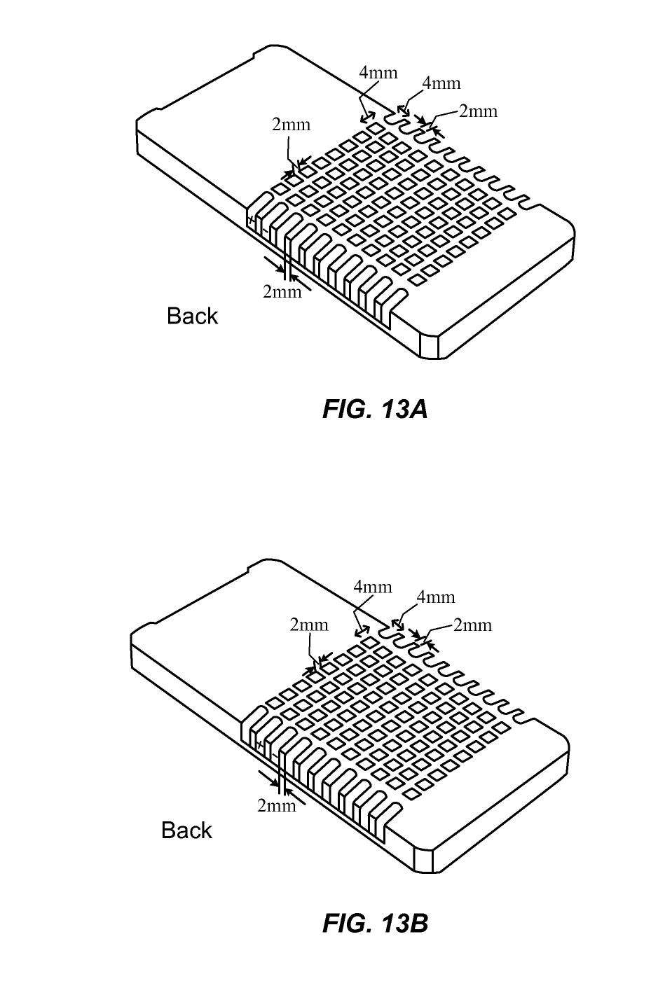

FIGS. 13A and 13B illustrate a handheld device having an interleaved electrode array layout on the back of the device, according to one example. As shown, interleaving positive and negative electrode pairs down the sides and on the back of the device can maximize an even distribution of electrodes and allow for an accurate single overall skin conductance measurement to be taken to capture a total electrodermal activity measurement when all the positive electrodes are fused together and all the negative electrodes are fused together, as described above. The maximized even distribution of electrodes may also allow for the skin conductance from each contacted pair contacted to be taken and then totaled or combined for a total skin conductance level result when an individual is resting the handheld device in his/her hand.

As shown, the back of the device may contain a plurality of rows and columns of electrodes that are approximately 4 mm×4 mm squares where each row and column of electrodes may be approximately 2 mm spaced apart on all sides, i.e. one square has a 2 mm gap around it. According to one example, making each electrode pair roughly the average size of a human fingertip may ensure an even contact area for positive and negative electrodes no matter how the device is contacted. As shown, only a single electrode pair is activated at any one time. Sampling each electrode pair automatically reverses the polarity. Furthermore, as described above, each electrode pair may be briefly scanned individually by sampling skin conductance for each pair, then adding the skin conductance from each contacted pair for a total result, then adjusting the threshold level based on number of pairs contacted to determine if an SCR occurred. Such a strategy may also automatically reverse the polarity of each electrode as each immediately adjacent electrode pair is individually scanned.

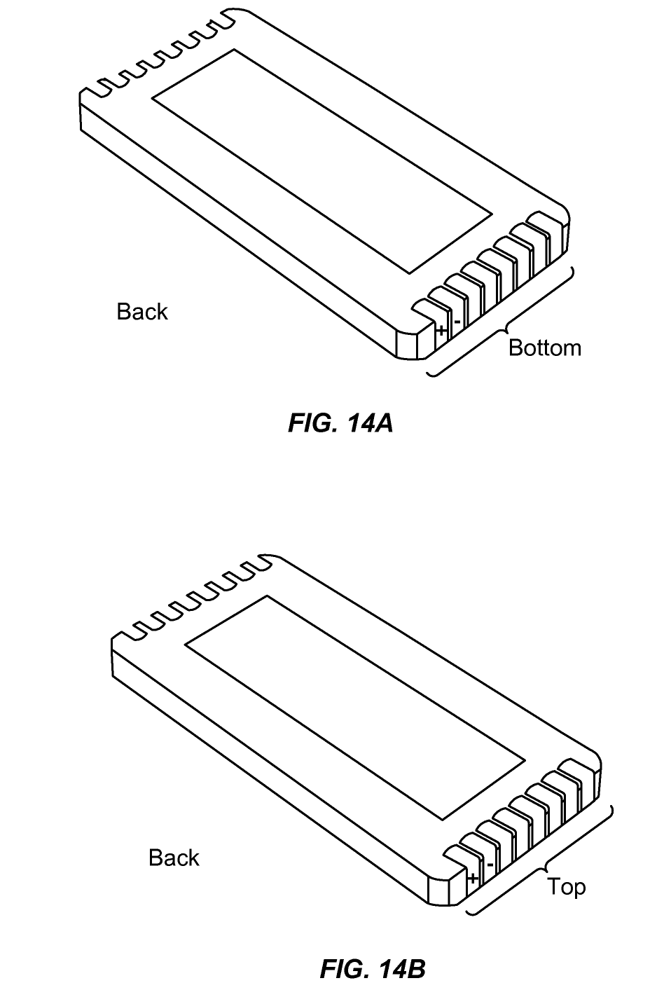

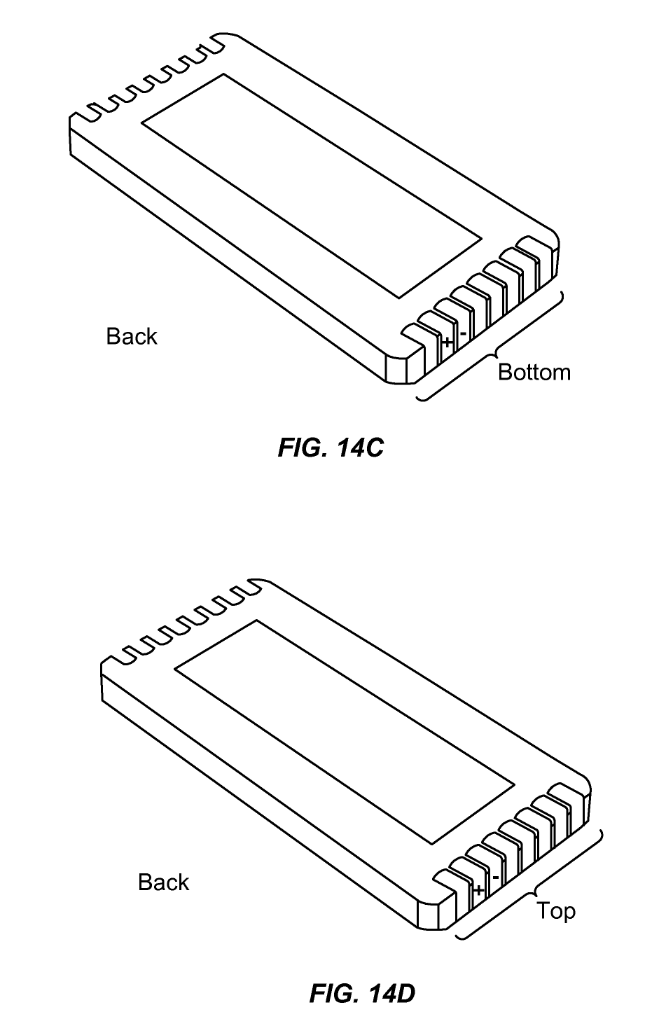

FIGS. 14A-14D illustrate a handheld device having an interleaved electrode array layout, according to one example. As shown, interleaving positive and negative electrode pairs on the top and bottom edge portions and wrapping around onto the back of the handheld device can maximize an even distribution of electrodes. Specifically, FIG. 14A illustrates a back view of a partial handheld device having an interleaved electrode array layout on a top edge portion, wrapping around to the backside, of the device sampling a first set of electrodes while FIG. 14B illustrates the back view of the partial handheld device of FIG. 14A, rotated 180 degrees, having an interleaved electrode array layout on a bottom edge portion, wrapping around to the backside, of the device sampling a first set of electrodes. FIG. 14C illustrates the back view of the partial handheld device of FIG. 14A sampling a second set of electrodes while FIG. 14D illustrates the back view of the partial handheld device of FIG. 14B sampling a second set of electrodes. An interleaved electrode array layout on the top and bottom edge portions that wrap around the backside of the device may be useful in measuring skin conductance if the individual is watching a video or playing a game on the device while the device is being held in the landscape mode.

As shown, interleaving positive and negative electrode pairs on the top and bottom portions of the device can maximize an even distribution of electrodes. According to one example, making each electrode pair roughly the average size of a human fingertip may ensure an even contact area for positive and negative electrodes no matter how the device is contacted. A single electrode pair may be activated at any one time. Sampling each electrode pair automatically reverses the polarity. Furthermore, as described above, each adjacent electrode pair may be briefly scanned individually by sampling skin conductance for each pair to determine which electrode pairs have been touched. Next, all the positive electrodes may be fused together and all the negative electrodes may be fused together and then one overall skin conductance measurement may be taken to capture total electrodermal activity measurement. The SCR threshold level may then be automatically adjusted based on number of electrodes contacted to determine if an SCR occurred. Alternatively, as described above, each adjacent electrode pair may be briefly measured individually by sampling skin conductance for each pair, then adding the skin conductance from each contacted pair for a total result, then adjusting the threshold level based on number of pairs contacted to determine if an SCR occurred. Such a strategy may also automatically reverse the polarity of each electrode as each immediately adjacent electrode pair is individually scanned.

### Grip Force:

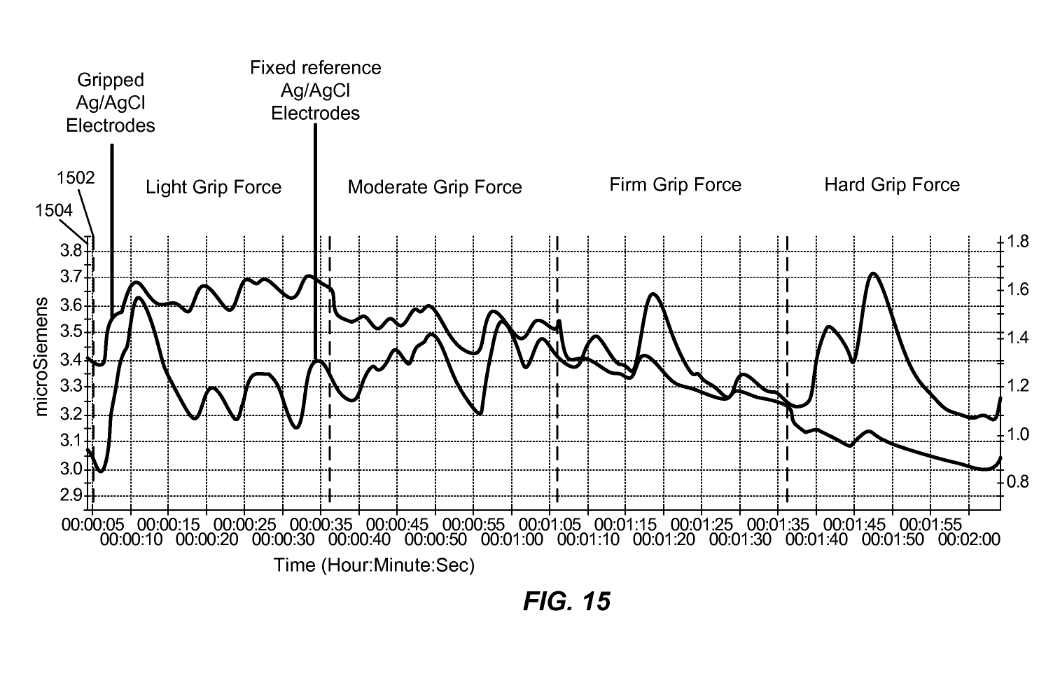

Grip force is the force that may temporarily be applied by an individual to the stainless steel electrodes on the handheld device. Changing grip force or applying too much grip force can result in distortion of the electrodermal signal on the handheld device which in turn may create false-positive and false-negative artifacts in the data. FIG. 15 is a graph illustrating the effects of various static grip forces applied to Ag/AgCl electrodes and on the skin conductance signal. The graph illustrates various static grip forces from light levels, to moderate levels, to firm levels and to hard levels applied to gripped Ag/AgCl electrodes and the resulting effects on the skin conductance signal on a first y-axis 1502 as compared to a fixed, worn reference skin conductance sensor on a second y-axis 1504. The grip force may be measured in microSiemens over a period of time in the format of hours, minutes, seconds. As shown in FIG. 15, the SCR amplitudes and skin conductance levels (SCL) may both decrease when the grip force exceeds some critical threshold (likely individually specific) which may result in false-negatives in the data. The example in FIG. 15 shows distortion of the skin conductance signal at firm and hard levels. This may be a result of firm to hard grips causing significant constriction of blood flow in an individual's hand. The constricted blood flow may result in decreased sweat production, the sweat operating as a transducer between ion flow in human sweat (NaCl) and electron flow in the circuit which allows skin conductance to be accurately calculated. For accurate measurement of skin conductance, some method of detecting grip force can be implemented to monitor when a critical grip force threshold has been exceeded. If a critical grip force has been exceeded, then skin conductance measurement could be stopped or the data invalidated.

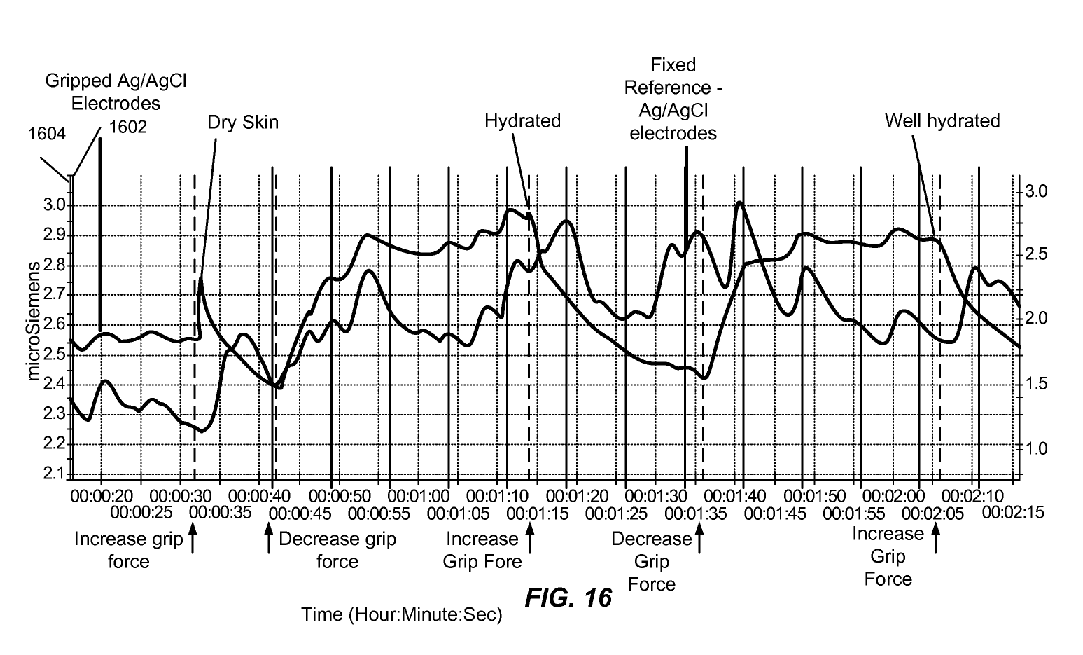

FIG. 16 is a graph illustrating the effects of dynamic grip force applied to Ag/AgCl electrodes and on the skin conductance signal. That is, shows the effects on electrodermal activity as changes to grip force occur on perfect electrodes. As shown in the graph, changing grip force can increase or decrease skin conductance depending on how dry (or hydrated) the skin is when applying grip force. The graph illustrates cycles of increasing the grip force from a moderate level to a firm level and then decreasing the grip force from a firm level to a moderate level. If the skin is dry and there is poor skin-electrode contact, increasing grip force could increase skin conductance as sweat may be squeezed out of the hand/fingers. If the skin is hydrated and there is good skin-electrode contact, increasing grip force may not change the signal at all if it is under the critical grip force threshold described above. If the applied force exceeds the critical threshold, skin conductance may in fact decline. Furthermore, it is possible that the act of changing grip force may improve the skin-electrode bond changing the effects of grip force on skin conductance. For accurate measurement of skin conductance some method of detecting grip force can be implemented to monitor when grip force is changing. If the grip forces changes significantly, then measurement of skin conductance could be stopped or the data invalidated. The graph illustrates various changes in grip force applied to gripped Ag/AgCl electrodes and the resulting effects on the skin conductance signal on a first y-axis 1602 as compared to a fixed, worn reference skin conductance sensor on a second y-axis 1604.

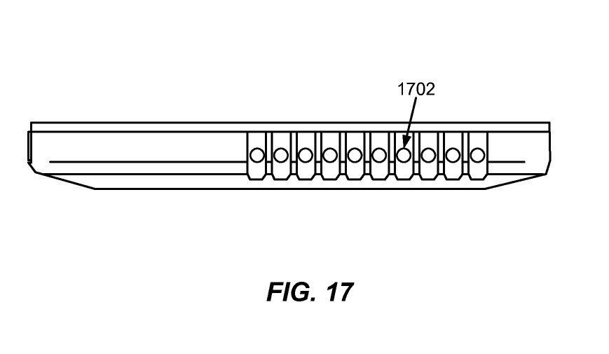

According to one embodiment, incorporating an array of force sensors, under the electrodermal electrode array, may allow changes in grip force to be captured and for static grip force to be monitored. Skin conductance data could be invalidated when grip force is changing or if grip force is greater than some critical threshold as determined in a calibration stage. FIG. 17 illustrates a side view of a handheld device showing force sensors 1702 placed directly under each of the electrodes. Although FIG. 17 illustrates force sensors placed directly under electrodes on the side of a handheld device, this is by way of example only and the force sensor may be placed directly under the electrodes arranged in different configurations such as FIGS. 13A-13B and FIGS. 14A-9D.

Exemplary Handheld Device and Operations Therein

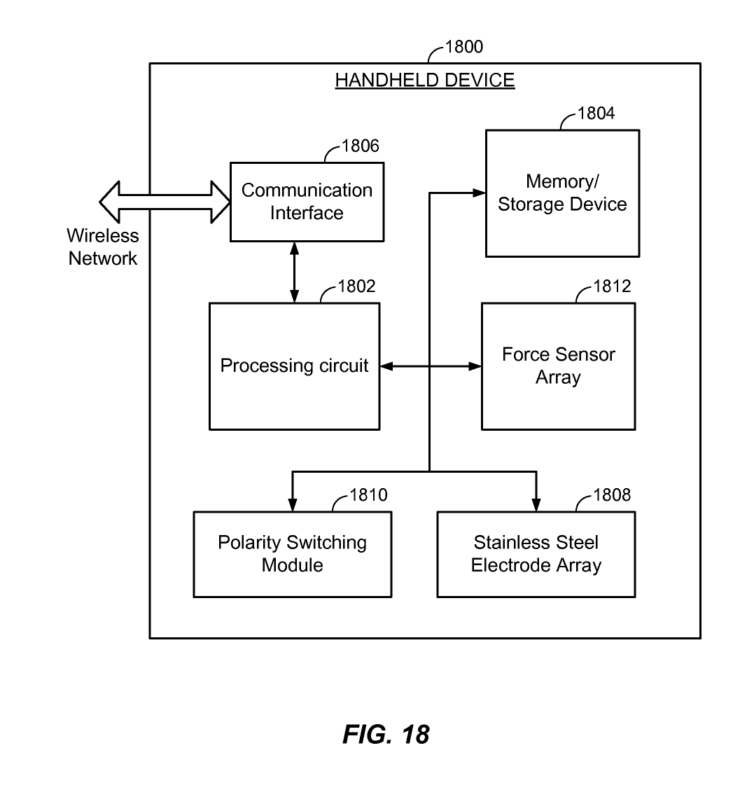

FIG. 18 illustrates a block diagram of an internal structure of a handheld device 1800, according to one example. The handheld device 1800 may include a processing circuit (e.g., processor, processing module, etc.) 1802 for executing computer-executable process steps and a memory/storage device 1804. The memory/storage device 1804 may include operations (instructions) for storing received input (or incoming) signals and/or feedback signals from electrodermal electrodes (i.e. electrodermal activity data).

The handheld device 1800 may also include a communication interface 1806 for communicatively coupling the handheld device 1800 to a wireless communication network as well as a stainless steel electrode array 1808 located on high contact locations, such as the side of the handheld device 1800. In one example, the stainless steel electrode array 1808 may include ten (10) curved electrode pairs on the sides of the handheld device 1800 so that equal portions of +/−electrodes may be contacted no matter how the device 1800 is gripped. In another example, the stainless steel electrode array 1808 may be an interleaved electrode array layout on the back of the handheld device. In yet another example, the stainless steel electrode array 1808 may include a plurality of electrodes located on the top and bottom edge portion wrapping around onto the back of a handheld device. The number of electrodes in the plurality of electrodes may vary with based on the length and/or width of the device. For example, the stainless steel electrode array 1808 may include ten (10) electrodes, one hundred (100) electrodes, less than ten (10) electrodes, between ten (10) electrodes and one hundred (100) electrodes or more than one hundred (100) electrodes. The electrodermal activity data from each pair of electrodes may be combined into a total electrodermal activity measurement. In one example, the electrodermal activity data may be in the form of a skin conductance signal and obtained by scanning each adjacent electrode pair, fusing the positive electrodes together and fusing the negative electrodes together and then taking one overall skin conductance measurement to capture total electrodermal activity measurement. In another example, the electrodermal activity data may be in the form of a skin conductance signal from each pair of electrodes and combining all the signals determines a total skin conductance level.

The handheld device 1800 may also include a polarity switching module 1810 coupled to the array of stainless steel electrodes 1808 embedded on the handheld device, for switching polarity of the electrode pairs in the array so that the current flow direction through the skin is reversed in a periodic manner at regular intervals. Additionally, an array of force sensors 1812 may be located under the array of electrode pairs 1808 for detecting grip force. If the grip force exceeds a threshold or the grip force is changing, the skin conductance measured may have artifacts and may not accurately reflect emotional arousal.

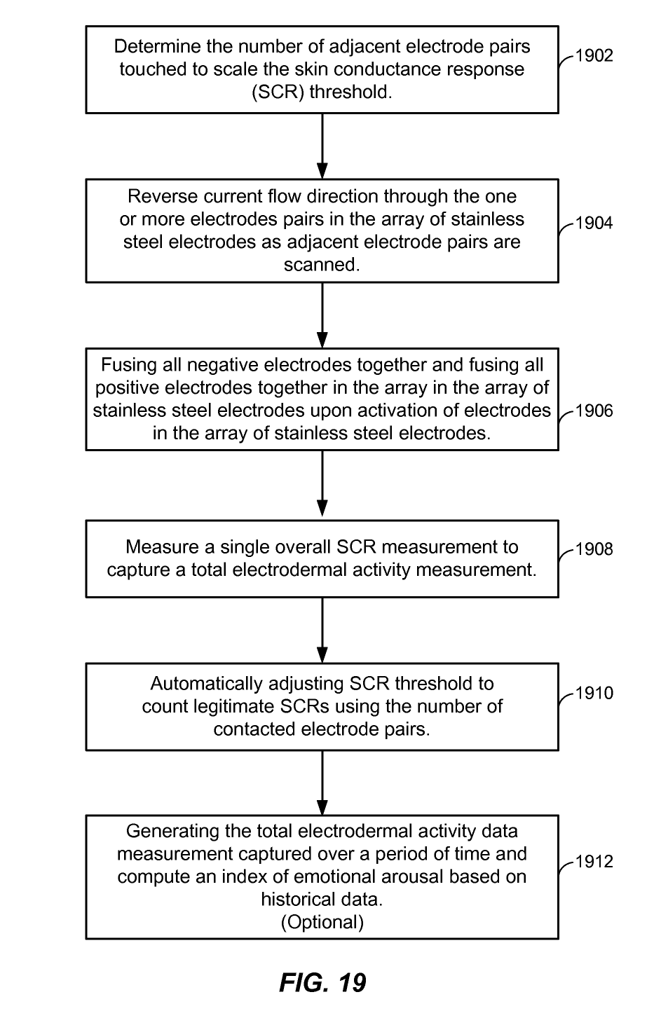

FIG. 19 illustrates a flow diagram of a method, which may be operational on a device, for acquiring electrodermal activity according to one example. Here, an array of stainless steel electrodes may be embedded on the sides and/or back of the device. Alternatively, the array of stainless steel electrodes may be embedded on top and bottom edge portions wrapping around to the backside of the device.

First, the number of electrode pairs that have been touched or gripped may be determined so that a skin conductance response (SCR) threshold can be scaled 1902. That is, the threshold may be adjusted depending on how many positive/negative electrode pairs are contacted at any point in time the device is gripped.

Next the current flow direction through the one or more electrode pairs in the array of stainless steel electrodes may be reversed as each adjacent electrode pair may be activated 1904. Next, all negative electrodes may be fused together and all positive electrodes may be fused together in the array of stainless steel electrodes upon activation of electrodes in the array of stainless steel electrodes 1906. The electrodes in the array of electrodes are activated upon the electrodes on the device becoming active and alternating in polarity, e.g. + − + − + − + − . Once all the positive electrodes are fused together and all the negative electrodes are fused together, a single (i.e. one) overall skin conductance measurement may be taken to capture a total electrodermal activity measurement 1908. The SCR threshold may then be automatically adjusted to count legitimate SCRs using the number of contacted electrode pairs 1910.

The total counted legitimate skin conductive responses may be a determination of the arousal of the individual. As SCR amplitude increases with increased surface area contacted, adapting the SCR threshold downwards may make it easier to find SCRs when only a few electrodes are touched than when many electrodes are touched. Optionally, the total electrodermal activity measurement captured over a period of time may be generated, in a graph for example, and an index of emotional arousal based on historical data may be computed 1912. An individual may then use this information in a biofeedback application for example to automatically adjust their skin conductance level to a lower value resulting in a more relaxed subjective state.

Alternatively, an index of arousal can be calculated based on the history or a individual's skin conductance data and fed into an application running on the device such as a game, social networking application or any other application running on the device that could make use of the individual's basic emotional status.

Changing grip force or grip force that is too great on the electrode pairs in the array of stainless steel electrodes can result in distortion of the electrodermal activity data on the handheld device which in turn may create false-positive and false-negative artifacts in the data. As such, independent of the electrode switching and scanning, to compensate for the possible false-positive and false-negative artifacts in the data, if the grip force is greater than a threshold or if the grip force is changing, captured electrodermal activity data may be invalidated.

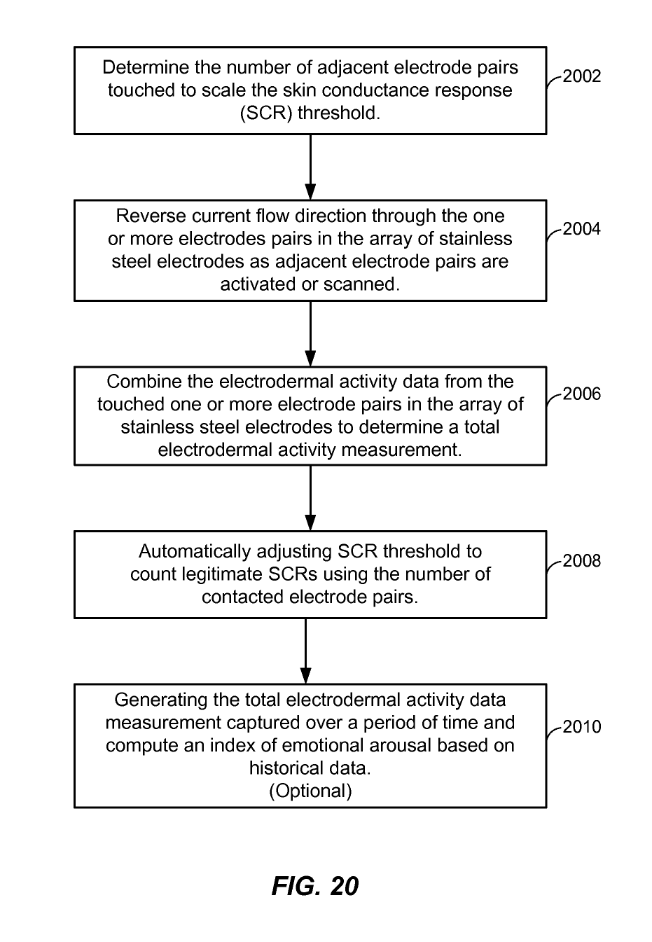

FIG. 20 illustrates a flow diagram of a method, which may be operational on a device, for acquiring electrodermal activity according to one example. Here, an array of stainless steel electrodes may be embedded on the sides and/or back of the mobile device. Alternatively, the array of stainless steel electrodes may be embedded on top and bottom edge portions wrapping around to the backside of the device.

First, the number of electrode pairs that have been touched or gripped may be determined so that a skin conductance response (SCR) threshold can be scaled 2002. That is, the threshold may be adjusted depending on how many positive/negative electrode pairs are contacted at any point in time the device is gripped.

Next the current flow direction through the one or more electrode pairs in the array of stainless steel electrodes may be reversed as each adjacent electrode pair may be activated 2004. The electrodermal activity data from the touched electrode pairs in the array of stainless steel electrodes may be combined to determine a total electrodermal activity measurement 2006. If the skin conductance level sampled exceeds some specified level (e.g., 0.1 microsiemens) then the electrode pair can be considered touched or contacted.

The total counted legitimate skin conductive responses may be a determination of the arousal of the individual. As SCR amplitude increases with increased surface area contacted, adapting the SCR threshold downwards may make it easier to find SCRs when only a few electrodes are touched than when many electrodes are touched. The SCR threshold may then be automatically adjusted to count legitimate SCRs using the number of contacted electrode pairs 2008.

Optionally, the total electrodermal activity measurement captured over a period of time may be generated, in a graph for example, and an index of emotional arousal based on historical data may be computed 2010. An individual may then use this information in a biofeedback application for example to automatically adjust their skin conductance level to a lower value resulting in a more relaxed subjective state.

Alternatively, an index of arousal can be calculated based on the history or an individual's skin conductance data and fed into an application running on the device such as a game, social networking application or any other application running on the device that could make use of the individual's basic emotional status.

Changing grip force or grip force that is too great on the electrode pairs in the array of stainless steel electrodes can result in distortion of the electrodermal activity data on the device which in turn may create false-positive and false-negative artifacts in the data. As such, independent of the electrode switching and scanning, to compensate for the possible false-positive and false-negative artifacts in the data, if the grip force is greater than a threshold or if the grip force is changing, captured electrodermal activity data may be invalidated.

In the foregoing specification, certain representative aspects of the invention have been described with reference to specific examples. Various modifications and changes may be made, however, without departing from the scope of the present invention as set forth in the claims. The specification and figures are illustrative, rather than restrictive, and modifications are intended to be included within the scope of the present invention. Accordingly, the scope of the invention should be determined by the claims and their legal equivalents rather than by merely the examples described.

For example, the steps recited in any method or process claims may be executed in any order and are not limited to the specific order presented in the claims. Additionally, the components and/or elements recited in any apparatus claims may be assembled or otherwise operationally configured in a variety of permutations and are accordingly not limited to the specific configuration recited in the claims.

Furthermore, certain benefits, other advantages and solutions to problems have been described above with regard to particular embodiments; however, any benefit, advantage, solution to a problem, or any element that may cause any particular benefit, advantage, or solution to occur or to become more pronounced are not to be construed as critical, required, or essential features or components of any or all the claims.

As used herein, the terms “comprise,” “comprises,” “comprising,” “having,” “including,” “includes” or any variation thereof, are intended to reference a non-exclusive inclusion, such that a process, method, article, composition or apparatus that comprises a list of elements does not include only those elements recited, but may also include other elements not expressly listed or inherent to such process, method, article, composition, or apparatus. Other combinations and/or modifications of the above-described structures, arrangements, applications, proportions, elements, materials, or components used in the practice of the present invention, in addition to those not specifically recited, may be varied or otherwise particularly adapted to specific environments, manufacturing specifications, design parameters, or other operating requirements without departing from the general principles of the same.

In one configuration, the interactive handheld device 1800 for acquiring electrodermal activity array of stainless steel electrodes embedded on the handheld device includes means for determining a number of adjacent electrode pairs in the array of stainless steel electrodes contacted to scale a skin conductance response threshold; means for fusing together negative and positive electrode pairs in the array of stainless steel electrodes; means for measuring a single overall skin conductance response to capture a total electrode activity measurement; means for detecting a grip force change from the temporary gripping of the one or more electrode pairs in the array of stainless steel electrodes; means for invalidating captured electrodermal activity data if changing grip force or if grip force exceeds a grip force threshold; and means for reversing reverse the current flow direction through the one or more electrode pairs in the array of stainless steel electrodes as each electrode pair is activated. In one aspect, the aforementioned means may be the processor(s) 1802 configured to perform the functions recited by the aforementioned means. In another aspect, the aforementioned means may be a module or any apparatus configured to perform the functions recited by the aforementioned means.

Moreover, in one aspect of the disclosure, the processing circuit 1802 illustrated in FIG. 18 may be a specialized processor (e.g., an application specific integrated circuit (ASIC)) that is specifically designed and/or hard-wired to perform the algorithms, methods, and/or steps described in FIGS. 19 and 20. Thus, such a specialized processor (ASIC) may be one example of a means for executing the algorithms, methods, and/or steps described in FIGS. 19 and 20. The memory circuit 1804 may also store processor 1802 readable instructions that when executed by a specialized processor (e.g., ASIC) of processor 1802 causes the specialized processor to perform the algorithms, methods, and/or steps described in FIGS. 19 and 20.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function.

Moreover, a storage medium may represent one or more devices for storing data, including read-only memory (ROM), random access memory (RAM), magnetic disk storage mediums, optical storage mediums, flash memory devices and/or other machine-readable mediums, processor-readable mediums, and/or computer-readable mediums for storing information. The terms “machine-readable medium”, “computer-readable medium”, and/or “processor-readable medium” may include, but are not limited to non-transitory mediums such as portable or fixed storage devices, optical storage devices, and various other mediums capable of storing, containing or carrying instruction(s) and/or data. Thus, the various methods described herein may be fully or partially implemented by instructions and/or data that may be stored in a “machine-readable storage medium”, “computer-readable storage medium”, and/or “processor-readable storage medium” and executed by one or more processors, machines and/or devices.

Furthermore, embodiments may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage(s). A processor may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The various illustrative logical blocks, modules, circuits, elements, and/or components described in connection with the examples disclosed herein may be implemented or performed with a general purpose processor, a digital signal processor (DSP), an application specific integrated circuit (ASIC), a field programmable gate array (FPGA) or other programmable logic component, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing components, e.g., a combination of a DSP and a microprocessor, a number of microprocessors, one or more microprocessors in conjunction with a DSP core, or any other such configuration.

The methods or algorithms described in connection with the examples disclosed herein may be embodied directly in hardware, in a software module executable by a processor, or in a combination of both, in the form of processing unit, programming instructions, or other directions, and may be contained in a single device or distributed across multiple devices. A software module may reside in RAM memory, flash memory, ROM memory, EPROM memory, EEPROM memory, registers, hard disk, a removable disk, a CD-ROM, or any other form of storage medium known in the art. A storage medium may be coupled to the processor such that the processor can read information from, and write information to, the storage medium. In the alternative, the storage medium may be integral to the processor.

Those of skill in the art would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system.

The various features of the invention described herein can be implemented in different systems without departing from the disclosure. It should be noted that the foregoing embodiments are merely examples and are not to be construed as limiting the invention. The description of the embodiments is intended to be illustrative, and not to limit the scope of the claims. As such, the present teachings can be readily applied to other types of apparatuses and many alternatives, modifications, and variations will be apparent to those skilled in the art.

What is claimed is:

1. A device, comprising:
an array of stainless steel electrodes;
a polarity switching module coupled to the array of stainless steel electrodes for switching polarity of electrodes in the array of stainless steel electrodes;
a memory device; and
at least one processor coupled to the array of stainless steel electrodes and the memory device, the at least one processor configured to:
determine a number of adjacent electrode pairs in the array of stainless steel electrodes contacted to scale a skin conductance response threshold;
fuse all negative electrodes together and all positive electrodes together in the array of stainless steel electrodes upon activation of the electrodes in the array of stainless steel electrodes; and
measure a single overall skin conductance response to capture a total electrode activity measurement.

2. The device of claim 1, further comprising a force sensor array coupled to each electrode pair in the array of stainless steel electrodes for detecting grip force.

3. The device of claim 2, wherein the at least one processor is further configured to:
invalidate captured electrodermal activity data if changing grip force or if the grip force exceeds a grip force threshold.

4. The device of claim 1, wherein the at least one processor is further configured to:
reverse the current flow direction through the one or more electrode pairs in the array of stainless steel electrodes as each electrode pair is activated.

5. The device of claim 1, wherein the total electrodermal activity measurement measures a reaction of an individual to an advertisement that has appeared on the device.

6. The device of claim 1, wherein the total electrodermal activity measurement is used to track stress levels of an individual.

7. The device of claim 6, wherein the at least one processor is further configured to:
generate a graph of the total electrodermal activity measurement captured over a period of time; and
compute an index of emotional arousal based on historical data.

8. The device of claim 1, wherein the at least one processor is further configured to:
automatically adjust the skin conductance response threshold to count legitimate skin conductive responses using the number of contacted electrode pairs, wherein the counted legitimate skin conductive responses is a determination of arousal.

9. The device of claim 1, wherein the array of stainless steel electrodes are embedded on a right side and a left side of the device.

10. The device of claim 1, wherein the array of stainless steel electrodes are interleaved down the sides and back of the device.

11. The device of claim 1, wherein the array of stainless steel electrodes are embedded on an upper edge portion and a lower edge portion wrapping around to a backside of the device.

12. The device of claim 1, wherein the device is an interactive handheld device.

13. A method for acquiring electrodermal activity on a device using an array of stainless steel electrodes embedded on the device, comprising:
determining a number of adjacent electrode pairs in the array of stainless steel electrodes contacted to scale a skin conductance response threshold;
fusing all negative electrodes together and all positive electrodes together in the array of stainless steel electrodes upon activation of electrodes in the array of stainless steel electrodes; and
measuring a single overall skin conductance response to capture a total electrode activity measurement.

14. The method of claim 13, further comprising detecting grip force from the temporary gripping of the one or more electrode pairs in the array of stainless steel electrodes.

15. The method of claim 14, further comprising invalidating captured electrodermal activity data if changing grip force or if the grip force exceeds a grip force threshold.

16. The method of claim 13, further comprising reversing the current flow direction through the one or more electrode pairs in the array of stainless steel electrodes as each electrode pair is activated.

17. The method of claim 13, wherein the total electrodermal activity measurement measures a reaction of an individual to an advertisement that has appeared on the device.

18. The method of claim 13, wherein the total electrodermal activity measurement is used to track stress levels of an individual.

19. The method of claim 18, further comprising:
generating a graph of the total electrodermal activity measurement captured over a period of time; and
computing an index of emotional arousal based on historical data.

20. The method of claim 13, further comprising automatically adjusting the skin conductance response threshold to count legitimate skin conductance responses using the number of contacted electrode pairs, wherein the counted legitimate skin conductive responses is a determination of arousal.

21. The method of claim 13, wherein the array of stainless steel electrodes are embedded on a right side and a left side of the device.

22. The method of claim 13, wherein the array of stainless steel electrodes are interleaved down the sides and back of the device.

23. The method of claim 13, wherein the array of stainless steel electrodes are embedded on an upper edge portion and a lower edge portion wrapping around to a backside of the device.

24. The method of claim 13, wherein the device is an interactive handheld device.

25. A device, comprising:
means for determining a number of adjacent electrode pairs in an array of stainless steel electrodes contacted to scale a skin conductance response threshold;
means for fusing all negative electrodes together and all positive electrodes together in the array of stainless steel electrodes upon activation of electrodes in the array of stainless steel electrodes; and
means for measuring a single overall skin conductance response to capture a total electrode activity measurement.

26. The device of claim 25, further comprising means for detecting a grip force change from the temporary gripping of the one or more electrode pairs in the array of stainless steel electrodes.

27. The device of claim 26, further comprising means for invalidating captured electrodermal activity data if changing grip force or if grip force exceeds a grip force threshold.

28. The device of claim 26, further comprising means for reversing the current flow direction through the one or more electrode pairs in the array of stainless steel electrodes as each electrode pair is activated.

29. The device of claim 26, wherein the total electrodermal activity measurement measures a reaction of an individual to an advertisement that has appeared on the device.

30. The device of claim 26, wherein the total electrodermal activity measurement is used to track stress levels of an individual.

31. The device of claim 30, further comprising:
means for generating a graph of the total electrodermal activity measurement captured over a period of time; and
means for computing an index of emotional arousal based on historical data.

32. The device of claim 26, further comprising means for automatically adjusting the skin conductance response threshold to count legitimate skin conductance responses using the number of contacted electrode pairs, wherein the counted legitimate skin conductive responses is a determination of arousal.

33. The device of claim 26, wherein the array of stainless steel electrodes are embedded on a right side and a left side of the device.

34. The device of claim 26, wherein the array of stainless steel electrodes are interleaved down the sides and back of the device.

35. The device of claim 26, the array of stainless steel electrodes are embedded on an upper edge portion and a lower edge portion wrapping around to a backside of the device.

36. The device of claim 26, wherein the device is an interactive handheld device.
